(19) United States
(12) Patent Application Publication
(10) Pub. No.: US 2004/0126414 A1
(43) Pub. Date: Jul. 1, 2004

(54) Methods and compositions for treating and preventing ear infections

(76) Inventor: Arthur F. Michaelis, Devon, PA (US)

Correspondence Address: CLARK & ELBING LLP, 101 FEDERAL STREET, BOSTON, MA, 02110, US

(21) Appl. No.: 10/734,338

(22) Filed: Dec. 11, 2003

Related U.S. Application Data
(60) Provisional application No. 60/433,428, filed on Dec. 12, 2002.

(51) Int. Cl. A61K 31/5415; A61L 15/16

(52) U.S. Cl. 424/446; 514/224.5

(57) ABSTRACT

The present invention relates to methods of treating, reducing, or preventing ear infections by topically administering a rifamycin of the invention to the ear of a patient. Infections amenable to treatment according to this invention include, for example, otitis media, otitis externa, or infections arising from surgery.

### CROSS REFERENCE TO RELATED APPLICATIONS

This application claims the benefit of the filing date of U.S. provisional application, U.S.S.No. 60/433,428, filed Dec. 12, 2002.

### BACKGROUND OF THE INVENTION

The invention relates to the field of bacterial infections of the ear.

Most ear infections are characterized by inflammation. In general, this condition, referred to as “otitis,” is treated as soon as it is diagnosed to reduce the risk of hearing loss, tinnitus, facial nerve palsy, mastoiditis, labyrinthitis, vertigo, and encephalitis.

The majority of ear infections affect either the external or the middle ear. Otitis externa (infection of the external ear) is primarily caused by bacterial infections (caused, for example, by Staphylococcus intermedius, Streptococcus spp., Pseudomonas spp., Proteus spp., and Escherichia coli). Normally, the external auditory canal is inhabited by a low concentration of bacteria, whose growth is largely inhibited by the slightly acidic pH and the build-up of cerumen (ear wax). Patients who scrape away the cerumen and epithelium leave an open wound characterized by a high pH, in turn establishing an environment favorable for bacterial infection. Furthermore, in patients whose ears are often submersed in water (due to swimming or sweating, for example), the skin swells and loses its natural acidic protection, therefore increasing the susceptibility of such patients to otitis externa. If untreated, infection of the external auditory canal may lead to inflammation of the middle and inner ear and may even spread to the pinna, periauricular soft tissues, or the temporal bone.

Otitis media, a common ailment in children, is a painful condition characterized by inflammation of the middle ear and resulting from a bacterial (e.g., Streptococcus pneumoniae, Haemophilis influenza, or Moraxella catarhalis) or viral infection. More than two-thirds of children in the United States have had at least one episode of otitis media by the age of three. Since otitis media is associated with significant childhood morbidity and is a primary cause of hearing loss in children, treatment of otitis media is critical.

Currently, the preferred method to treat both otitis media and otitis externa is the administration of antibiotics. The unresponsiveness of patients to antibiotics has progressively increased in recent years due to the emergence of antibiotic-resistant bacterial strains. Although amoxicillin, for example, is a preferred antibiotic used to treat otitis media, one-third of Haemophilis influenzae strains and at least three-quarters of Moraxella catarhalis strains are β-lactamase producers and are therefore inherently resistant to this antibacterial agent. In instances in which children are infected with such resistant strains, the administration of more potent antibiotics is required but these treatments often cause life-threatening responses.

Thus, there is a need for improved methods for treating ear infections.

### SUMMARY OF THE INVENTION

In general, the present invention features methods and compositions for treating, reducing, or preventing an ear infection in a patient by topically administering to the affected otic area (e.g., the tympanic membrane or the external auditory canal of the ear) of the patient a pharmaceutical composition containing a therapeutically effective amount of a rifamycin of the invention. The compositions and methods of the invention are also useful to treat, reduce, or prevent infections that result from surgery. Patients may therefore be administered with the composition of the invention prior to and following the surgical procedure.

Accordingly, the present invention features a pharmaceutical composition containing a rifamycin (e.g., rifalazil) and a pharmaceutically-acceptable excipient. Desirably, this composition is suitable for topical administration to the ear of a patient. According to this invention, the patient being treated is administered with a dose of rifamycin in an amount sufficient to treat, reduce, or prevent an infection in the patient. The rifamycin may be present in the composition in an amount ranging between 0.001% and 5% weight/volume (w/v), preferably between 0.01% and 3% w/v, more preferably between 0.1% and 1% w/v, or most preferably between 0.1% and 0.4% w/v. Optionally, the rifamycin may be impregnated in a porous media (for example, an ear wick such as a sponge, gauze, cotton, or hydrocellulose), which is suitable for insertion into the ear of a patient. The composition may also include one or more penetration enhancers (e.g., alcohols, polyols, sulfoxides, esters, ketones, amides, oleates, surfactants, alkanoic acids, lactam compounds, alkanols, or admixtures thereof). If desired, a second therapeutic agent may also be admixed with the composition of the invention. Exemplary therapeutic agents useful for the present invention include, for example, anti-inflammatory agents (e.g., non-steroidal anti-inflammatory or steroid), anesthetics (benzocaine, butamben picrate, tetracaine, dibucaine, prilocaine, etidocaine, mepivacaine, bupivicaine, and lidocaine), zinc salts (. Zinc salts include zinc sulfate, zinc chloride, zinc acetate, zinc phenol sulfonate, zinc borate, zinc bromide, zinc nitrate, zinc glycerophosphate, zinc benzoate, zinc carbonate, zinc citrate, zinc hexafluorosilicate, zinc diacetate trihydrate, zinc oxide, zinc peroxide, zinc salicylate, zinc silicate, zinc stannate, zinc tannate, zinc titanate, zinc tetrafluoroborate, zinc gluconate, and zinc glycinate), or antimicrobial agents (amoxillin, erythromycin, azithromycin, clarithromycin, gentamicin, tobramycin, ciprofloxaxin, norfloxacin, gatifloxacin, ofloxacin, levofloxacin, moxifloxacin, metronidazole, lomefloxacin, ciprofloxacin, natamycin, neomycin, polymyxin B, gentamycin, trovafloxacin, grepafloxacin, sulfacetamide, tetracycline, gramicidin, chloremphenicol, bacitracin, and gramicidin). Non-steroidal anti-inflammatory agents include, for example, detoprofen, diclofenac, diflunisal, etodolac, fenoprofen, flurbiprofen, indomethacin, ketoprofen, mechlofenameate, mefenamic acid, meloxicam, nabumeone, naproxen sodium, oxaprozin, piroxicam, sulindac, tolmeting, celecoxib, rofecoxib, choline salicylate, salsate, sodium salicylate, magnesium salicylate, aspirin, ibuprofen, paracetamol, acetaminophen, and pseudoephedrine and steroids include, for example, hydrocortisone, prednisone, fluprednisolone, triamcinolone, dexamethasone, betamethasone, cortisone, prednilosone, methylprednisolone, fluocinolone acetonide, flurandrenolone acetonide, and fluorometholone.

The invention also features a method of treating, preventing, or reducing an ear infection in a patient using any of the compositions described above. Ear infections that may be treated using the methods and compositions of the invention include otitis media (e.g., acute otitis media, otitis media with effusion, and chronic otitis media) and otitis externa (e.g., acute otitis externa, chronic otitis externa, and malignant otitis externa). According to the present invention, the rifamycin is administered to the ear (e.g., the tympanic membrane or the external auditory canal of the ear) to treat or prevent bacterial infections associated with otitis media (e.g., Haemophilus influenza, Moraxella catarhalis, or Streptococcus pneumoniae) or otitis externa (e.g., Staphylococcus intermedius, Streptococcus spp. Pseudomonas spp., Proteus spp or Escherichia coli). The rifamycin may be administered to the infected ear by means of drops or by the insertion of a rifamycin-impregnated porous media into the external ear canal to the tympanic membrane.

The methods and compositions of the invention are also useful to treat, prevent, or reduce infections associated with otic surgical procedures, including for example, tympanoplasty, stapedectomy, removal of tumors, or cochlear implant surgery. Treatment using the compositions described herein may therefore be used as a prophylactic measure in anticipation of therapies or conditions that may cause ear infections. Accordingly, compositions containing the rifamycin may be applied to an area of the ear to which the surgical intervention will be performed, within at least seven days, five days, four days, three days, two days, one day, six hours, two hours, or one hour (before or after) of the surgical intervention. When treating a patient affected with otitis externa, an acidification therapy involving the administration of an acetic acid solution to the ear of the patient may also be performed.

Typically, patients are administered one to four drops of the composition of the invention, which contains a rifamycin in a total amount ranging between 0.001% and 5% w/v, preferably between 0.01% and 3% w/v, more preferably between 0. 1% and 1% w/v, or most preferably between 0.1% and 0.4% w/v. The composition may be given daily (e.g., once, twice, three times, or four times daily) or less frequently (e.g., once every other day, or once or twice weekly). Treatment may last for 1 to 21 days, desirably 1 to 14 days, or even 3 to 7 days. If desired, second therapeutic agents, such as anti-inflammatory agents (e.g., non-steroidal anti-inflammatory or steroid), anesthetics, zinc salts, or other antimicrobial agents, may also be administered with the rifamycin of the invention. Such additional therapeutic agents may be administered in the same or in a different pharmaceutical composition as the rifamycin. If the therapeutic agent is present in a different pharmaceutical composition as the rifamycin, different routes of administration may be used. The rifamycin and the second therapeutic agent may be administered within two days, 24 hours, six hours, three hours, or one hour of each other. In addition to being administered topically, the second therapeutic agent may also be administered orally or by injection (e.g., intravenously, intramuscularly, or subcutaneously).

To increase the efficacy of the topically administered rifamycin-containing composition, it is desirable that the amount of debris and granulation tissue be reduced in the infected ear of the patient by at least one hour prior to the administration of the rifamycin and at least once a day. Debris may be removed by any known method such as by suction, by irrigation with a solution containing hydrogen peroxide, by cauterization, or by manual techniques employing microinstruments and microscope. Reduction in the amount of granulation tissue in the infected ear may be performed by cautering or by the administration of a steroid.

The invention also features a pharmaceutical pack containing (i) a rifamycin in an amount effective to treat a patient having an ear infection; and (ii) instructions for administering the rifamycin to the ear of a patient. The invention also features a container containing a rifamycin of the invention and a pharmaceutical excipient suitable for topical administration to the ear. If desired, an applicator for applying the composition to the ear may also be provided with the container. The rifamycin may be present in amounts ranging between 0.001% and 5% weight/volume (w/v), preferably between 0.01% and 3% w/v, more preferably between 0.1% and 1% w/v, or most preferably between 0.1% and 0.4% w/v. Desirably, the rifamcyin is present in amounts sufficient to treat a patient for at least 1, 3, 5, 7, 10, 14, or 21 days. A penetration enhancer may also be added (e.g., alcohols, polyols, sulfoxides, esters, ketones, amides, oleates, surfactants, alkanoic acids, lactam compounds, alkanols, or admixtures thereof).

By “an effective amount” is meant the amount of a rifamycin of the invention required to result in the reduction, treatment, or prevention of an ear infection. The presence of bacteria may be determined by a diagnostic test that detects for presence of such bacteria in a bacterial culture obtained, for example, by tympanocentesis. Prophylactic administration of a rifamycin of the invention is considered to be preventing the development of an ear infection.

An ear infection has been treated when one or more tests of the disease (e.g., any standard methods known in the art such as those described below) indicate that the patient's condition has improved. An infection may be detected by a pneumatic otoscopic examination of the patient or by a reduction in infection-associated symptoms in the patient (e.g., inflammation of ear drums, redness of ear drums, or presence of fluid in ears). Reduction of symptoms may also be determined, for example, using an audiogram to check recovery from hearing loss.

By “debris” is meant the mucoid exudate or desquamated epithelium in an infected ear of a patient.

By “ear wick” is meant a sponge, cotton, gauze, compressed hydroxycellulose, or any other material used to increase the penetration of rifamycin to the infected otic area. The ear wick is typically inserted into the canal under direct vision. Its presence helps wick eardrops along the canal, hold the solution in contact with the skin of the canal, and apply pressure to the canal skin.

By “granulation tissue” is meant the highly vascularized tissue that replaces the initial fibrin clot in a wound. Vascularization is a result of an ingrowth of capillary endothelium from the surrounding vasculature. The tissue is also rich in fibroblasts and leucocytes.

Rifamycins include rifalazil, rifampin, rifabutin, rifapentin, rifaximin, and compounds described by formula I:

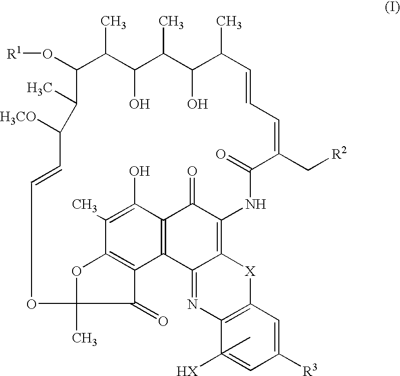

In formula I, X represents an oxygen atom or a sulfur atom, R¹ represents a hydrogen or an acetyl group, R² represents a hydrogen or hydroxyl group, and R³ represents a group expressed by the formula:

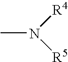

such that each of R⁴ and R⁵ is, independently, an alkyl group having 1 to 7 carbon atoms, or R⁴ and R⁵ combine to form a 3-8 membered cyclic system. Alternatively, R³ represents a group expressed by the formula:

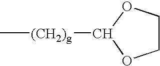

in which g represents an integer between 1 and 3;

R³ may also be represented by a group expressed by the formula:

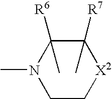

such that each of R⁶ and R⁷ is, independently, a hydrogen atom or an alkyl group having 1 to 3 carbon atoms and X² represents an oxygen atom, a sulfur atom, or a carbonyl group. X² may also represent a group expressed by the formula:

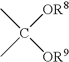

in which each of R⁸ and R⁹ is, independently, a hydrogen atom or an alkyl group having 1 to 3 carbon atoms. R⁸ and R⁹ may also, in combination with each other, represent —(CH₂)ₖ— in which k represents an integer between 1 and 4. X² may alternatively represent a group expressed by the formula:

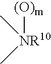

in which m represents 0 or 1, R¹⁰ represents a hydrogen atom, an alkyl group having 1 to 6 carbon atoms, or —(CH₂)ₙX³ in which n represents an integer between 1 and 4 and X³ represents an alkoxy group having 1 to 3 carbon atoms, a vinyl group, an ethynyl group. X² may further represent a group expressed by the formula:

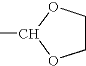

An exemplary rifamycin of the invention is rifalazil (3′-hydroxy-5′-(4-isobutyl-1-piperazinyl) benzoxazinorifamycin) also known as KRM-1648. Other rifamycins of the invention are disclosed in U.S. Pat. Nos. 4,690,919; 4,983,602; 5,786,349; 5,981,522; 6,316,433 and 4,859,661, each of which is hereby incorporated by reference.

### DETAILED DESCRIPTION OF THE INVENTION

Ear infections typically affect the middle or the external ear and include, for example, otitis media, otitis externa, and infections caused by surgical interventions. Due to secondary complications that arise from ear infections (e.g., hearing loss), the treatment and prevention of such conditions is critical.

We have discovered that topical administration of a rifamycin of the invention is effective in treating, preventing, or reducing an infection of the ear, such as otitis media or otitis externa. In the case of otitis media or otitis externa, infections are primarily caused by Haemophilus influenza, Moraxella catarhalis, Streptococcus pneumoniae, Streptococcus pyogenes, Staphylococcus intermedius, Staphylococcus epidermidis, Staphylococcus aureus, Staphylococcus caprae, Staphylococcus auriculis, Staphylococcus capitis, Staphylococcus haemolytis, Pseudomonas aeroginosa, Proteus mirabilis, Proteus vulgaris, Escherichia faecalis, or Escherichia coli. The rifamycins of the invention are used to treat infections of the ear caused by any of these bacteria. The methods and compositions of the invention are also useful to treat, reduce, or prevent infections of the ear resulting from surgical interventions. The rifamycin may therefore be administered topically to the area of the ear to which surgical intervention was performed or alternatively, the rifamycin may be administered to the ear of the patient prophylactically, that is, prior to otic surgery, noninvasive otic procedures, or other types of surgery. Exemplary surgical procedures include for example, cochlear implant surgery, tympanoplasty, tympanostomy tube insertion, removal of tumors (e.g., cholesteatoma), or stapedectomy. The rifamycin may be administered to the area of the ear to which surgical intervention will be performed, for example, within seven days, two days, one day, 12 hours, 10 hours, 6 hours, 4 hours, 2 hours, 1 hour, or less than 1 hour prior to or following the surgical intervention. The compositions may be used for acute treatment of temporary conditions, or may be administered chronically.

The rifamycin may be given daily (e.g., once, twice, three times, or four times daily) or less frequently (e.g., once every other day, or once or twice weekly). Typically, patients are administered a dosage of one to four drops of a solution containing the rifamycin. The compound may be contained in any appropriate amount in any suitable carrier substance, and is generally present in an amount ranging between 0.001% and 5%, desirably between 0.01% and 3%, more desirably between 0.1% and 1%, and even more desirably between 0.1% and 0.4% by weight of the total volume (w/v) of the composition. The rifamycin is provided in a dosage form that is suitable for topical administration. Thus, a rifamycin-containing composition may be in the form of a solution, aerosol, gel, ointment, nebulizer, or suspension. Alternatively, the rifamycin may be administered to the patient being treated by placing a rifamycin-impregnated porous media into the external ear canal to the tympanic membrane. The pharmaceutical composition can generally be formulated according to conventional pharmaceutical practice (see, e.g., Remington: The Science and Practice of Pharmacy (20th ed.), Ed. A. R. Gennaro, 2000, Lippincott Williams & Wilkins, Philadelphia, and Encyclopedia of Pharmaceutical Technology, Eds. J. Swarbrick and J. C. Boylan, 1988-1999, Marcel Dekker, New York).

Aural Toilet

In an infected ear, the external auditory canal and tissues lateral to the infected middle ear often are frequently covered with mucoid exudate or desquamated epithelium. Since topically applied preparations cannot generally penetrate affected tissues until these interposing materials are removed, aural toilet is desirably performed before administering the rifamycin of the invention. Aural toilet may be performed by a health provider, the patient, or any other individual. Removal of debris may be performed mechanically with the assistance of a microscope and microinstruments. Aural irrigation may also be performed using a solution containing peroxide. The concentration of peroxide used for this purpose should be the highest concentration that may be applied without causing any significant pain or discomfort to the patient. As an example, a solution of 50% peroxide and 50% sterile water may be used. 30 to 40 mL of this solution is irrigated through the external auditory canal using a small syringe or bulb-type aspirator. The irrigant solution is allowed to drain out (e.g., for 5-10 minutes) prior to administering a rifamycin of the present invention.

Granulation Tissue

Granulation tissue often fills the middle ear and medial portions of the external auditory canal and reducing this accumulation is beneficial for resolution of an ear infection. Granulation tissue may also prevent topically applied antimicrobial agents from penetrating to the site of infection and desirably, the amount of granulation tissue is reduced prior to and throughout the regimen. Although topical antimicrobial drops may reduce granulation by eliminating infection and by removing the inciting irritating inflammation, the amount of granulation tissue may be reduced using other methods known in the art. For example, topical steroids may hasten the resolution of middle ear granulation, thus improving penetration of topically delivered antibiotics.

Cautery may also be used to reduce the amount and the formation of granulation tissue. Microbipolar cautery may be administered by a health provider. Chemical cautery using silver nitrate, for example, may also be applied to an infected ear in the form of silver nitrate sticks. Excision of granulation tissue may also be performed by a health care provider using a microscope and microinstruments.

Ear Canal Acidification

In a patient affected with otitis externa, a therapy involving ear canal acidification to restore the physiological acidity of the ear may be performed. The affected ear may be administered with a solution containing acetic acid, which may also include a steroid (e.g., hydrocortisone), aluminum acetate, or rubbing alcohol.

Topical Formulations

Pharmaceutical compositions according to the present invention are desirably formulated for topical administration to the ear of the patient. Patients having an ear infection may be administered with effective amounts of the rifamycin of the invention, by means of a solution (e.g., drops), ointment, gel, or aerosol (e.g., nebulizer). The composition is typically administered to the affected otic area by topically applying one to four drops of a solution or suspension or a comparable amount of an ointment, gel, or other solid or semisolid composition, once, twice, three times, or more than three times per day. A porous media or an ear wick (e.g., cotton, gauze, or compressed hydroxycellulose) may also be used to increase the penetration of the rifamycin to the infected otic area. The ear wick, which is inserted into the canal under direct vision, is typically a dried sponge that helps wick eardrops along the canal, hold the solution in contact with the skin of the canal, and apply pressure to the canal skin. Wicks may be removed one day, two days, or more than two days following insertion, and may be replaced if necessary. Alternatively, the ear wick may itself be impregnated with the rifamycin. These formulations can be made according to known and conventional methods for preparing such formulations.

Since some of the rifamycins of this invention are not highly soluble in water at physiological conditions, a solubilizing excipient may be used to increase solubility. Solubilization is taken to mean an improvement in the solubility by virtue of surface-active compounds that can convert substances that are insoluble or virtually insoluble in water into clear, or opalescent, aqueous solutions without changing the chemical structure of these substances in the process. Excipients used for this purpose are restricted to those that are safe for administration to humans. Typically such co-solvents are employed at a level of about 0.01% to 2% by weight.

A variety of solubilizing excipients may be used for the formulation of the rifamycin, including compounds belonging to the following classes: polyethoxylated fatty acids, PEG-fatty acid diesters, PEG-fatty acid mono-ester and di-ester mixtures, polyethylene glycol glycerol fatty acid esters, alcohol-oil transesterification products, polyglycerized fatty acids, propylene glycol fatty acid esters, mixtures of propylene glycol esters and glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-polyoxypropylene block copolymers, sorbitan fatty acid esters, lower alcohol fatty acid esters, or ionic surfactants. Such excipients are described for example, in U.S. Patent Application No. 60/385,532, hereby incorporated by reference.

Ototopical preparations may vary in viscosity. The use of viscosity enhancing agents to provide the compositions of the invention with viscosities greater than the viscosity of simple aqueous solutions may be desirable to increase the retention time in the ear. Such viscosity-building agents include, for example, polyvinyl alcohol, polyvinyl pyrrolidone, methyl cellulose, hydroxypropyl methylcellulose, hydroxyethyl cellulose, carboxymethyl cellulose, hydroxypropyl cellulose, or other agents known to those skilled in the art. Such agents are typically employed at a level of about 0.01% to 2% by weight. Optionally, these preparations may include a buffering agent to maintain an acidic pH since the normal environment of the external auditory canal is acidic. However, if treatment is required in the middle ear where the pH is neutral, the pH may be adjusted accordingly.

Otic pharmaceutical products are typically packaged in multidose form. Preservatives are thus desired to prevent microbial contamination during use. Suitable preservatives include: polyquaternium-1, benzalkonium chloride, thimerosal, chlorobutanol, methyl paraben, propyl paraben, phenylethyl alcohol, edetate disodium, sorbic acid, or other agents known to those skilled in the art. Typically, such preservatives are employed at a level of from 0.001% to 1.0% by weight.

A penetration enhancer may also be used to facilitate the diffusion of the rifamycin through the tympanic membrane into the middle and inner ear in order to reduce inflammation of ear tissues. A penetration enhancer is an agent used to increase the permeability of the skin to a pharmacologically active agent to increase the rate at which the drug diffuses through the skin and enters the tissues and bloodstream. A chemical skin penetration enhancer increases skin permeability by reversibly damaging or by altering the physiochemical nature of the stratum corneum to reduce its diffusional resistance (Osborne D W, Henke J J, Pharmaceutical Technology, November 1997, pp 58-86). Examples of penetration enhancers include without limitation: alcohols, such as ethanol and isopropanol; polyols, such as n-alkanols, limonene, terpenes, dioxolane, propylene glycol, ethylene glycol, other glycols, and glycerol; sulfoxides, such as dimethylsulfoxide (DMSO), dimethylformamide, methyl dodecyl sulfoxide, dimethylacetamide; esters, such as isopropyl myristate/palmitate, ethyl acetate, butyl acetate, methyl proprionate, and capric/caprylic triglycerides; ketones; amides, such as acetamides; oleates, such as triolein; various surfactants, such as sodium lauryl sulfate; various alkanoic acids, such as caprylic acid; lactam compounds, such as azone; alkanols, such as oleyl alcohol; dialkylamino acetates, and admixtures thereof. The use of such penetration enhancers is disclosed, for example, in U.S. Pat. No. 6,093,417, hereby incorporated by reference.

Other Therapeutic Agents

As discussed below, preparations containing the rifamycin of the present invention may also include a second therapeutic agent, including for example, another rifamycin, an anesthetic, an antimicrobial agent, a zinc salt, or an anti-inflammatory agent (e.g., an non-steroidal anti-inflammatory or a steroid). When admixing an anti-microbial agent, the antimicrobial agent is preferably amoxillin, erythromycin, azithromycin, clarithromycin, gentamicin, tobramycin, ciprofloxaxin, norfloxacin, gatifloxacin, ofloxacin, levofloxacin, moxifloxacin, metronidazole, lomefloxacin, ciprofloxacin, natamycin, neomycin, polymyxin B, gentamycin, bacitracin, trovafloxacin, grepafloxacin, sulfacetamide, tetracycline, gramicidin, chloremphenicol, or gramicidin. Preferred non-steroidal anti-inflammatory agents include, for example, detoprofen, diclofenac, diflunisal, etodolac, fenoprofen, flurbiprofen, indomethacin, ketoprofen, mechlofenameate, mefenamic acid, meloxicam, nabumeone, naproxen sodium, oxaprozin, piroxicam, sulindac, tolmeting, celecoxib, rofecoxib, choline salicylate, salsate, sodium salicylate, magnesium salicylate, aspirin, ibuprofen, paracetamol, acetaminophen, and pseudoephedrine, and preferred steroids include, for example, hydrocortisone, prednisone, fluprednisolone, triamcinolone, dexamethasone, betamethasone, cortisone, prednilosone, methylprednisolone, fluocinolone acetonide, flurandrenolone acetonide, and fluorometholone. Preferred anesthetics according to the invention include, for example, benzocaine, butamben picrate, tetracaine, dibucaine, prilocaine, etidocaine, mepivacaine, bupivicaine, and lidocaine. Useful zinc salts include zinc sulfate, zinc chloride, zinc acetate, zinc phenol sulfonate, zinc borate, zinc bromide, zinc nitrate, zinc glycerophosphate, zinc benzoate, zinc carbonate, zinc citrate, zinc hexafluorosilicate, zinc diacetate trihydrate, zinc oxide, zinc peroxide, zinc salicylate, zinc silicate, zinc stannate, zinc tannate, zinc titanate, zinc tetrafluoroborate, zinc gluconate, and zinc glycinate. All of the therapeutic agents employed in the compositions of the present invention may be used in the dose ranges currently known and used for these agents. Different concentrations may be employed depending on the clinical condition of the patient, the goal of therapy (treatment or prophylaxis), the anticipated duration, and the severity of the infection for which the rifamycin of the invention is being administered. Additional considerations in dose selection include the type of infection, age of the patient (e.g., pediatric, adult, or geriatric), general health, and comorbidity.

### OTHER EMBODIMENTS

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in microbiology or related fields are intended to be within the scope of the invention.

What is claimed is:

1. A pharmaceutical composition comprising a compound of formula (I): 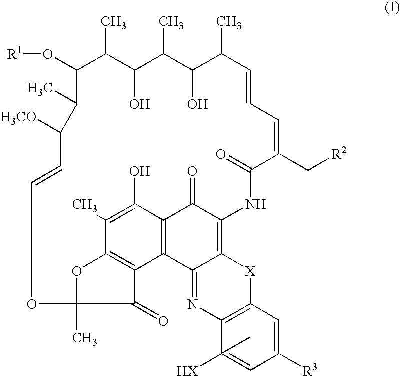
in an amount between 0.001% and 5% weight/volume (w/v) and a pharmaceutically acceptable carrier suitable for topical administration to the ear of a patient:

2. The pharmaceutical composition of claim 1, wherein said compound is rifalazil.

3. The pharmaceutical composition of claim 1, further comprising a porous media impregnated with said compound, said media being suitable for insertion into the ear of said patient.

4. The pharmaceutical composition of claim 2, wherein said porous media is an ear wick comprising a substrate selected from the group consisting of a sponge, gauze, cotton, and hydroxycellulose.

5. The pharmaceutical composition of claim 1, wherein said composition further comprises a penetration enhancer.

6. The pharmaceutical composition of claim 7, wherein said penetration enhancer is selected from the group consisting of alcohols, polyols, sulfoxides, esters, ketones, amides, oleates, surfactants, alkanoic acids, lactam compounds, alkanols, and admixtures thereof.

7. The pharmaceutical composition of claim 1, further comprising a second therapeutic agent.

8. The pharmaceutical composition of claim 7, wherein said second therapeutic agent is an anesthetic, anti-inflammatory agent, anti-microbial agent, or zinc salt.

9. A method for treating or preventing a bacterial ear infection in a patient, said method comprising topically administering to the ear of said patient a compound of formula (I): 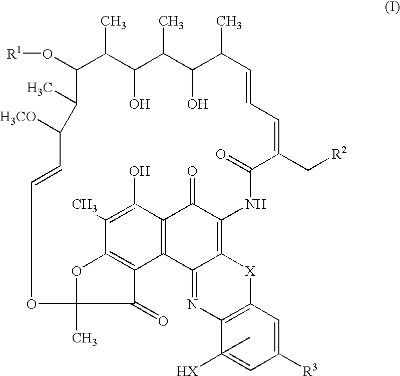
in an amount effective to treat or prevent said ear infection in said patient.

10. The method of claim 9, wherein said compound is rifalazil.

11. The method of claim 9, wherein said patient is a human.

12. The method of claim 9, wherein said ear infection is otitis media or otitis externa.

13. The method of claim 9, wherein said composition is administered to the tympanic membrane or external auditory canal of said patient.

14. The method of claim 9, wherein said patient has undergone or will undergo surgery of said ear.

15. The method of claim 14, wherein said composition is administered to the area of the ear to which surgery has been or will be performed.

16. The method of claim 15, wherein said composition is administered within seven days prior to and after said surgery.

17. The method of claim 9, wherein said patient is administered one to four drops of a pharmaceutical composition comprising said compound, wherein said compound is in the amount between 0.001% and 5% w/v.

18. The method of claim 9, wherein said compound is administered for a period of 1 to 14 days.

19. The method of claim 9, wherein said method further comprises administering to said patient a second therapeutic agent.

20. The method of claim 19, wherein said second therapeutic agent is an anesthetic, anti-inflammatory agent, anti-microbial agent, or zinc salt.

21. The method of claim 19, wherein said compound and said second therapeutic agent are administered in the same formulation.

22. The method of claim 19, wherein said compound and said second therapeutic agent are administered in different formulations.

23. The method of claim 19, wherein said compound and said second therapeutic agent are administered by different routes of administration.

24. The method of claim 19, wherein said compound and said second therapeutic agent are administered within 24 hours of each other.

25. The method of claim 9, wherein said method further comprises reducing the amount of debris or granulation tissue in said ear of said patient prior to said topical administration of said compound.

26. A pharmaceutical pack comprising (i) a composition comprising a compound of formula (I): 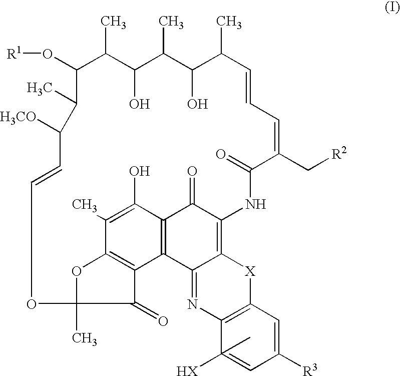
in an amount effective to treat a patient having an ear infection; and (ii) instructions for administering said compound to the ear of said patient.

27. The pharmaceutical pack of claim 26, wherein said compound is in an amount between 0.001% and 5% w/v.

28. The pharmaceutical pack of claim 26, wherein said compound is rifalazil.

29. A container comprising a composition comprising a compound of formula (I): 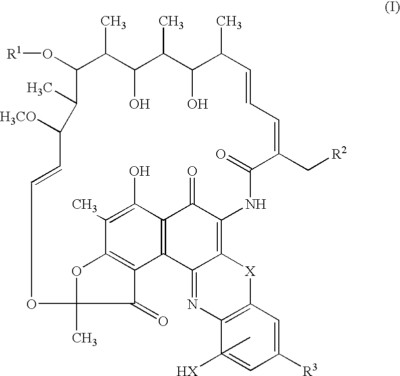
and a pharmaceutical excipient suitable for topical administration to the ear of a patient.

30. The container of claim 29, wherein the container further comprises an applicator for applying said composition to the ear.
